(19) United States
(12) Patent Application Publication
(10) Pub. No.: US 2026/0240822 A1
(43) Pub. Date: Aug. 20, 2026

(54) Methods and Compositions for Treating Atopic Dermatitis

(71) Applicant: Regranion, LLC, Mt. Pleassant, SC (US)

(72) Inventor: Gautam S. GHATNEKAR, Mt. Pleasant, SC (US)

(21) Appl. No.: 19/443,669

(22) Filed: Jan. 8, 2026

Related U.S. Application Data
(63) Continuation of application No. PCT/US24/23850, filed on Apr. 10, 2024.
(60) Provisional application No. 63/525,788, filed on Jul. 10, 2023.

(51) Int. Cl. A61K 31/437; A61K 9/00; A61P 17/00

(52) U.S. Cl. CPC A61K 31/437; A61K 9/0053; A61P 17/00

(57) ABSTRACT

The present invention provides compositions and methods for treating atopic dermatitis. In one embodiment, the invention provides a method or a compound for treating atopic dermatitis in a subject in need thereof. The method comprises administering to the subject a therapeutically effective amount of Compound I,

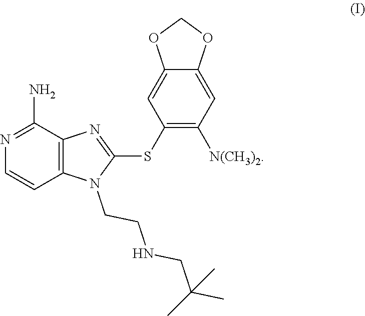 [SMILES: CN(C)c1cc2c(cc1Sc1nc3c(N)nccc3n1CCNCC(C)(C)C)OCO2]

### RELATED APPLICATIONS

This application is a continuation of International Application No. PCT/US24/23850 which designated the United States and was filed on Apr. 10, 2024, published in English, which claims the benefit of U.S. Provisional Application No. 63/525,788, filed on Jul. 10, 2023. The entire teachings of the above applications are incorporated herein by reference.

### BACKGROUND

Heat shock protein (HSP) 90 is a common chaperone that folds and supports the activity of many proteins including key proinflammatory proteins. Hence, HSP90 inhibition may be a wide-ranging approach targeting different inflammatory pathways, representing a novel mechanism of action for treating inflammatory skin diseases.

Tukaj et al. Cell Stress and Chaperones (2019) 24:475-479 and Tukaj et al. Biomolecules (2022) 12, 1153 review heat shock proteins as potential treatment targets in the therapy of autoimmune diseases.

Atopic dermatitis (AD) is a common inflammatory skin disorder characterised by dry and itchy skin that imposes a high disease burden on patients and the healthcare system, affecting up to 20% of children and up to 5% of adults.¹⁻³ AD is associated with atopic comorbidities (e.g., food allergy, allergic rhinitis and asthma), but other comorbidities have been reported including cardiovascular and neuropsychiatric disorders.^{4,5} Although the multifactorial pathophysiology remains to be fully explored, the mechanisms underlying AD implicate a complex interaction of components involving skin barrier dysfunction, immune dysregulation, altered skin microbiome and genetic predisposition.⁶ Recent molecular insights into the disease mechanisms have revealed significant pathways and novel targets, enabling the vast ongoing drug development and the recent approval of therapeutics targeting IL-4, IL-13 and JAK1/2.⁷ Nonetheless, some groups of patients do not achieve satisfactory long-term control or tolerate current treatments, highlighting the pressing need for novel treatments.

Therefore, there is a need for new compositions and methods for treating AD.

### SUMMARY

The present invention provides compositions and methods for treating atopic dermatitis. In one embodiment, the invention provides a method for treating atopic dermatitis in a subject in need thereof, comprising administering to the subject a therapeutically effective amount of Compound I,

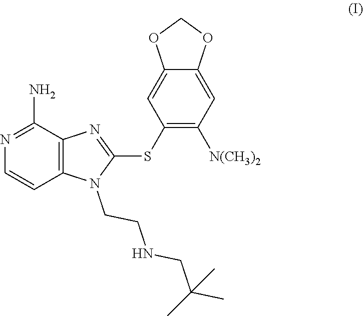 [SMILES: CN(C)c1cc2c(cc1Sc1nc3c(N)nccc3n1CCNCC(C)(C)C)OCO2]

or a pharmaceutically acceptable salt thereof.

The invention further provides Compound I, or a pharmaceutically acceptable salt thereof, for use in treating atopic dermatitis.

The invention further provides a pharmaceutical composition comprising Compound I, or a pharmaceutically acceptable salt thereof, for treating atopic dermatitis.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIG. 1 shows RT-qPCR analysis of AD-related gene expression in lesional/non-lesional AD skin and cytokine-stimulated primary human keratinocytes. Effects of Compound I (5 μM) or dexamethasone (0.1 μM) on inflammatory gene expression in primary human keratinocytes stimulated with TNF (10 ng/ml) in combination with IFNγ (10 ng/ml) or IL-4 (100 ng/ml) for 24 hours (three independent experiments, n=6). Data are shown as mean±SEM. *p<0.05, **p≤0.01, ***p<0.001, ****p≤0.0001. ns, not significant. Abbreviations: Dexa, dexamethasone.

FIG. 2 shows the effects of Compound I (5 μM) or dexamethasone (0.1 μM) on the phosphorylation status of key signalling proteins determined by Western blot in primary human keratinocytes stimulated with TNF (10 ng/ml) in combination with (A) IFNγ (10 ng/ml) or (B) IL-4 (100 ng/mL) for 1 hour (three independent experiments, n=6-9). Representative Western blots and densitometric results (mean±SEM) are shown. *p<0.05, **p<0.01, ***p<0.001, ****p≤0.0001. ns, not significant. Abbreviations: Dexa, dexamethasone.

FIG. 3 illustrates ear thickness changes throughout the experiments and photographs on day 9 in MC903-induced atopic dermatitis mouse model. Female Balb/cAnNRj ears were challenged daily with (A) 1 nmol or (B-C) 2.5 nmol of MC903 for 6 days and treated once daily with drug-vehicle, topical dexamethasone (20 nmol), topical compound of Compound I (1 mg) or oral compound of Compound I (100 mg/kg). Four to eight mice in each group, in total 76 mice. Two independent experiments were conducted. Data are shown as mean±SEM. *p<0.05, **p≤0.01, ***p≤0.001, ****p≤0.0001. ns, not significant. Statistical significance denotes pair-wise comparison on day 9; Black, MC903+Drug-vehicle vs. Vehicle; Blue, MC903+compound of Compound I vs. MC903+Drug-vehicle; Teal, MC903+ compound of Compound I vs. MC903+Dexa. Dexa, dexamethasone.

FIG. 4 presents the results of histological analysis of ear biopsies from mice challenged daily with 1 nmol of MC903 and treated with drug-vehicle, topical compound of Compound I or topical dexamethasone. (A) Epidermal thickness, (B) dermal thickness and (C) total number of cells were determined from haematoxylin & eosin (HE) stained sections. (D) Quantification of mast cells in toluidine blue stained sections. (E) Quantification of Lyg6+ cells (neutrophils) and (F) CD4+ cells (T cells) in immunohistochemical stained sections. (G) Representative images of stained sections. Data are shown as mean±SEM. *p<0.05, **p<0.01, ****p≤0.0001. ns, not significant. Abbreviations: Dexa, dexamethasone.

FIG. 5 shows RT-qPCR (upper panels) and RNA sequencing analysis (lower panels) of AD-related cytokine gene expression in ear tissue from mice challenged daily with 1 nmol of MC903 and treated with drug-vehicle, topical compound of Compound I or topical dexamethasone. Data are shown as mean±SEM. *p<0.05, **p≤0.01, ***p≤0.001, ****p≤0.0001. ns, not significant.

FIG. 6 shows the results of RNA sequencing analysis of ear tissue from mice challenged daily with 1 nmol of MC903 and treated with drug-vehicle, topical compound of Compound I or topical dexamethasone. (A) Principal component analysis (PCA) of regularized log (rlog) transformed data. (B) Heatmap of differentially expressed genes (DEGs; log 2[foldchange]>1.5 and FDR-adjusted p-value<0.05) upon MC903-induced inflammation. The genes were filtered to include those with a mean of normalised counts >25. (C-D) Volcano plots for two different comparisons. The dashed horizontal lines mark an FDR-adjusted p-value of 0.05, whereas the vertical lines mark a log 2(fold change) of 1.5. DEGs are marked as red dots. (E-F) Enrichment analyses of down-regulated DEGs by compound of Compound I compared with drug-vehicle; (E) the top ten significant GO biological processes terms; (F) five relevant significantly enriched KEGG pathway terms are presented. Abbreviations: Dexa, dexamethasone. PCA, Principal component analysis.

FIG. 7 is a graph showing the concentrations of Compound I found in biopsied tissues in subjects as described in Example 2.

### DETAILED DESCRIPTION

The present invention relates to methods and a compound for treating atopic dermatitis. In one embodiment, the invention provides a method of treating atopic dermatitis in a human subject in need thereof, comprising administering to the subject a therapeutically effective amount of Compound I or a pharmaceutically acceptable salt thereof. The invention further relates to Compound I, or a pharmaceutically acceptable salt thereof, for use in treating atopic dermatitis.

Compound I is a potent inhibitor of HSP90, and the synthesis and HSP90 inhibitory activity of Compound I are described in WO2008/115719, the contents of which are incorporated by reference herein in their entirety. Compound I is also known as CUDC-305, Debio-0932, and RGRN-305.

In the methods and compounds for treating atopic dermatitis of the invention, the subject is preferably a human subject. The subject can have atopic dermatitis with symptoms at any degree of severity. For example, the subject can have an EASI Score of 0 indicating clear or no eczema present; a score of 0.1 to 1.0 indicates almost clear; a score of 1.1 to 7 indicates mild disease, 7.1 to 21 indicates moderate disease, a score of 21.1 to 50 indicates severe disease, and a score of greater than 51 indicates very severe disease.

Compound I or a pharmaceutically acceptable salt thereof is preferably administered in the form of a pharmaceutical composition comprising the therapeutic agent and a pharmaceutically acceptable carrier, excipient or diluent. Suitable pharmaceutical compositions include a solid, semisolid or liquid preparation (tablet, pellet, troche, capsule, suppository, cream, ointment, aerosol, powder, liquid, emulsion, suspension, syrup, injection, etc.). The pharmaceutical composition can be administered by any suitable means, including, without limitation, parenteral, intravenous, intramuscular, subcutaneous, implantation, oral, sublingual, buccal, nasal, pulmonary, transdermal, topical, vaginal, rectal, and transmucosal administrations or the like. Topical administration can involve the use of transdermal administration such as transdermal patches or iontophoresis devices.

In a preferred embodiment, the pharmaceutical composition is administered orally, for example, as a solid or a liquid preparation. Suitable solid oral formulations include tablets, capsules, pills, granules, pellets, sachets and effervescent, powders, and the like. Suitable liquid oral formulations include solutions, suspensions, dispersions, emulsions, oils and the like. In one embodiment of the present invention, the composition is formulated in a capsule. In another embodiment, the composition is formulated as a tablet. Compound I shows unexpectedly high accumulation in the skin following oral dosing.

Any inert excipient that is commonly used as a carrier or diluent may be used in the pharmaceutical compositions of the present invention, such as for example, a gum, a starch, a sugar, a cellulosic material, an acrylate, or mixtures thereof. A preferred diluent is microcrystalline cellulose. The compositions may further comprise a disintegrating agent (e.g., croscarmellose sodium) and a lubricant (e.g., magnesium stearate), and may additionally comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof. Furthermore, the compositions of the present invention may be in the form of controlled release or immediate release formulations.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCl., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, polyethylene glycol), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In one embodiment, the active compound is prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art, for example, as described in U.S. Pat. No. 4,522,811.

It is especially advantageous to formulate oral compositions in unit dosage form for ease of administration and uniformity of dosage. “Unit dosage form”, as used herein, refers to physically discrete units suited as unitary dosages for the subject to be treated; each unit containing a predetermined quantity of active compound calculated to produce the desired therapeutic effect in association with the required pharmaceutical carrier. The specifications for the unit dosage forms of the invention are dictated by and directly dependent on the unique characteristics of the active compound and the particular therapeutic effect to be achieved, and the limitations inherent in the art of compounding such an active compound for the treatment of individuals.

Formulations of the invention intended for oral administration can include one or more permeation enhancers, including long chain fatty acids or salts thereof, such as decanoic acid and sodium decanoate.

The pharmaceutical compositions can be included in a container, pack, or dispenser together with instructions for administration.

The pharmaceutical composition of the invention is preferably suitable for oral administration and is most preferably in the form of a tablet. In one embodiment, the pharmaceutical composition is a tablet comprising Compound I. In certain embodiments the tablet comprises Compound I in the form of the free base. In certain embodiments, the tablet comprises Compound I free base in an amount from 0.5 mg to 500 mg, from 1 mg to 450 mg, from 10 mg to 350 mg, from 20 mg to 300 mg, from 40 mg to 275 mg, 50 mg to 260 mg or 250 mg. Alternatively, the tablet comprises a pharmaceutically acceptable salt of Compound I in the foregoing amounts where these amounts represent free base equivalents. Preferably, the tablet comprises 0.5 mg, 1 mg, 5 mg, 20 mg, 50 mg, 100 mg, 125 mg or 250 mg of Compound I free base.

The pharmaceutical composition of the invention is preferably suitable for topical administration and is most preferably in the form of a cream, a lotion, a gel, an ointment, an aerosol, a powder, a liquid, an emulsion, a suspension, or via transdermal patches or via iontophoresis devices. In one embodiment, the pharmaceutical composition is a cream comprising Compound I. In certain embodiments, the cream comprises Compound I in the form of the free base. In certain embodiments, the cream comprises Compound I free base in an amount from 0.25 mg to 250 mg, from 0.5 mg to 225 mg, from 1 mg to 200 mg, from 1.5 mg to 175 mg, from 2 mg to 150 mg, from 2.5 mg to 125 mg, from 3 mg to 100 mg, from 4 mg to 75 mg, from 4.5 mg to 50 mg, from 5 mg to about 25 mg. Preferably, the cream comprises 0.5 mg, 1 mg, 5 mg, 20 mg, 50 mg, 100 mg, 125 mg or 250 mg of Compound I free base.

The pharmaceutical composition can be administered daily or on a suitable schedule. In one embodiment, daily administration is repeated continuously for a period of several days to several years. Oral treatment may continue for between one week and the life of the patient. The administration can be continuous or intermittent, e.g., treatment for a number of consecutive days followed by a rest period.

The amount of the compound administered to the patient is preferably less than an amount that would cause toxicity in the patient. In certain embodiments, the amount of Compound I or pharmaceutically acceptable salt thereof that is administered to the patient is less than the amount that causes a concentration of the compound in the patient's plasma to equal or exceed the toxic level of the compound.

The dosing schedules described herein describe administration of Compound I, which can be administered in the form of the free base or as a pharmaceutically acceptable salt. Any amount of Compound I administered in the dosing regimens described herein refers the amount of the free base form, or with respect to a pharmaceutically acceptable salt, the free base equivalent amount.

In certain embodiments, Compound I is administered at a dose of 0.5 mg to 400 mg per dosing day. In certain embodiments, Compound I is administered at a dose of 10 mg to 50, 100 or 200 mg per dosing day. In certain embodiments, Compound I is administered orally to the subject at a dose of 0.5 mg to 300 mg per dosing day, 25 mg to 275 mg per dosing day, 30 mg to 260 mg per dosing day or 250 mg per dosing day. In certain embodiments, Compound I is administered orally to the subject at a dose of 0.5 mg to 200 mg per dosing day, 20 mg to 175 mg per day, 20 mg to 150 mg per dosing day, 20 mg to 125 mg per dosing day, 20 mg to 100 mg per dosing day, or 20 mg to 50 mg per dosing day. In certain embodiments, Compound I is administered orally to the subject at a dose of 50 mg to 150 mg per dosing day, 60 mg to 140 mg per dosing day, 70 mg 130 mg per dosing day, 75 mg to 125 mg per dosing day, 80 mg to 120 mg per dosing day, 90 mg to 110 mg per dosing day or 100 mg per dosing day.

In certain embodiments, Compound I is administered orally to the subject at a dose of 150 mg to 300 mg per dosing day, 200 mg to 300 mg per dosing day, 225 mg to 275 mg per dosing day, 240 mg to 260 mg per day, 245 to 255 mg/day or 250 mg per dosing day.

The term “dosing day”, as used herein, refers to a day on which Compound I, in the form of the free base or a pharmaceutically acceptable salt, is administered to the subject. In certain embodiments, Compound I is administered to the subject daily. In other embodiments, Compound I is administered every other day. In certain embodiments, Compound I is administered to the subject three days per week, preferably on non-consecutive days, for example on Monday, Wednesday and Friday. The dose mentioned is the amount of drug per day and will correlate to the concentration of drug in the specific dosage format and the amount/area applied.

In certain embodiments, Compound I is initially administered to the subject at a loading dose of 0.5-250 mg per dosing day for a suitable period of time, such as 1 to 12 weeks, 2 to 10 weeks or 4 to 8 weeks. The loading dose is preferably administered daily. The loading dose can be followed by a reduced maintenance dose, for example any of the reduced dosing schedules and/or reduced doses per dosing day as described above. For example, Compound I can be administered at loading dose of 25-250 mg/dosing day for 4 to 8 weeks, followed by a maintenance dose of 0.5-200 mg per dosing day, provided that the maintenance dose is a lower dose per dosing day and/or dosed less frequently than the loading dose. In certain embodiments, the loading dose is 200 to 250 mg per day for 4 to 8 weeks and the maintenance dose is 25 to 200 mg per day, provided that the maintenance dose is lower than the loading dose.

Dosing of Compound I or a pharmaceutically acceptable salt thereof as described above can continue for any period of time and is preferably continued for at least 16 weeks or more. Preferably dosing continues as long as the subject is exhibiting clinical improvement in the signs and symptoms of atopic dermatitis.

Dosing of Compound I or a pharmaceutically acceptable salt thereof as described above can be interrupted by occasional periods of one or more days in which dosing is suspended, and then dosing can be continued either on a predetermined scheduled or upon return or worsening of atopic dermatitis signs and symptoms.

Response of a subject to the therapeutic methods of the present invention can be determined as is known in the art. For example, the subject can be evaluated according to the Physician's Global Assessment (PGA) and/or Dermatology Life Quality Index (DLQI).

### Definitions

Listed below are definitions of various terms used to describe this invention. These definitions apply to the terms as they are used throughout this specification and claims, unless otherwise limited in specific instances, either individually or as part of a larger group.

Compound I can be formulated and administered in the methods of the invention as the free base or as a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977). The salts can be prepared in situ during the final isolation and purification of the compound of the invention, or separately by reacting the free base function with a suitable organic acid or inorganic acid. Examples of pharmaceutically acceptable nontoxic acid addition salts include, but are not limited to, salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid lactobionic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

As used herein, “pharmaceutically acceptable carrier” is intended to include any and all solvents, dispersion media, coatings, antibacterial and antifungal agents, isotonic and absorption delaying agents, and the like, compatible with pharmaceutical administration, such as sterile pyrogen-free water. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated.

The term “subject” as used herein refers to an animal. Preferably the animal is a mammal. Most preferably, the subject is a human.

The terms “therapeutically effective amount” and “effective amount” of a therapeutic agent refers to an amount of such agent which confers a therapeutic effect on the treated subject, at a reasonable benefit/risk ratio applicable to treatment of atopic dermatitis. A therapeutically effective amount of an agent may be different when used as a single agent that when used in combination with one or more other agents. In addition, a therapeutically effective amount of an agent may depend on the specific combination of agents to be administered. The therapeutic effect may be objective (i.e., measurable by some test or marker) or subjective (i.e., subject gives an indication of or feels an effect). Therapeutically effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound or composition of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of symptoms.

### EXAMPLES

### Example 1—In Vitro and In Vivo Studies of Compound I Relevant to Atopic Dermatitis Cell Culture and Experiments

Primary normal human epidermal keratinocytes (NHEKs) were isolated from nine healthy donors as described previously.¹³ The keratinocytes were plated in 6-well plates and cultured in keratinocyte SFM (Gibco, ThermoFisher Scientific, Waltham, MA, USA) supplemented with growth factors and 5 μg/ml Gentamicin (Gibco) at 37° C. in a humidified incubator containing 5% CO2 until 60-70% confluency. Subsequently, the keratinocytes were starved for 24 hours in keratinocyte SFM medium without growth factors before the initiation of experiments. After 2 hours of preincubation with vehicle (0.2% ethanol diluted in water), Compound I (5 μM) or dexamethasone (0.1 μM; Sigma-Aldrich, Burlington, MA, USA), the keratinocytes were stimulated with TNF (10 ng/ml; PeproTech, London, UK) in combination with IFNγ (10 ng/mL; PeproTech) or IL-4 (50 ng/mL; PeproTech) for up to 24 hours to induce an AD-like phenotype.¹⁴⁻¹⁶ The used concentration of Compound I was based on previous concentration studies demonstrating anti-inflammatory properties and low toxicity (3-6%) in stimulated NHEKs.^{8,10}

### Human Samples

Four-millimetres punch biopsies from six AD patients were collected from lesional and non-lesional skin and stored in liquid nitrogen until RNA isolation.

RNA Isolation from NHEKs

Keratinocytes were washed with phosphate-buffered saline (PBS; Gibco) followed by RNA extraction using SV 96 Total RNA Isolation System (Promega, Madison, WI, USA) per the manufacturers' instructions. NanoDrop 2000 Spectrophotometer (ThermoFisher) was used to determine the RNA concentration and purity.

RNA Isolation from Punch Biopsies

Punch biopsies were stored in 750 μL RNAlater-ICE (ThermoFisher) at −80° C. for 20 minutes and overnight at −20° C. Then, the biopsies were added to 175 μL SV RNA lysis buffer with β-mercaptoethanol (SV Total RNA Isolation System; Promega) followed by homogenization using TissueLyser (Qiagen, Hilden, Germany). The remaining steps including RNA purification and DNase treatment were conducted following the manufacturer's instructions.

### Reverse Transcription-Quantitative PCR

cDNA was generated using total RNA, TaqMan Reverse Transcription Reagents with random hexamers (ThermoFisher) and Peltier Thermal Cycler-200 (MJ Research Inc, Waltham, MA, USA) following the manufacturer's instructions. Real-time PCR was carried out with 20 ng cDNA per 20 μL reaction in StepOnePlus Real-Time PCR system (ThemoFisher) using TaqMan Universal PCR Master Mix, primers and probes (ThermoFisher) for TNF (Hs00174128_m1), ILIB (Hs01555410_m1), IL6 (Hs00174131_m1), TSLP (Hs00263639_m1), CCL17 (Hs00171074_m1), CCL22 (Hs01574247_m1), Il1b (Mm00434228_m1), Il4 (Mm00445259_m1), Il6 (Mm00446190_m1), Il13 (Mm00434204_m1) RPLPO (Hs99999902_m1) and Gapdh (Mm99999915_m1). Three technical replicates for each sample underwent real-time PCR; 2 minutes at 50° C. and 10 minutes at 95° C. followed by 40 cycles of 15 seconds at 95° C. and 1 minute at 60° C. The standard curve method with StepOne Software v2.1 and reference genes (RPLPO and Gapdh) were used to obtain normalised relative expression levels of target genes.¹⁷

Protein Isolation from NHEKs

PBS-washed keratinocytes were added cell lysis buffer with complete Protease Inhibitor Cocktail and phenylmethylsulphonyl fluoride (Sigma-Aldrich). The lysate was centrifuged at 13,000 g for 3 minutes followed by collection of the supernatant containing the protein extract. The protein concentration was determined by Bradford Protein Assay.

### Western Blot

A total of 20 μg protein extract for each sample was loaded on 10% mini-PROTEAN TGX PreCast Gel (Bio-Rad, Hercules, CA, USA) and separated by gel electrophoresis using Mini Trans-Blot Cell (Bio-Rad). The subsequent protein blotting onto nitrocellulose membranes was performed with Trans-Blot Turbo Transfer System (Bio-Rad). Membranes were incubated overnight at 4° C. with the following primary antibodies from Cell Signaling Technology Danvers, MA, USA (catalog #): P-STAT1 (#9167), P-STAT 3 (#9145) P-P65 (#3033), and P-STAT6 (#56554). After washing, the membranes were incubated with HRP-conjugated anti-rabbit IgG antibody (Cell Signaling Technology; catalog #7074) at room temperature for 1 hour. Protein bands were detected with an enhanced chemiluminescence (ECL) reaction using Clarity Western ECL Substrate (Bio-Rad) and digitally imaged with C-DiGit Blot Scanner (LI-COR, Lincoln, NE, USA). To normalize the protein expression to β-actin levels, the membranes were stripped and reprobed with an anti-β-actin antibody (A1978; Sigma-Aldrich), which was detected by HRP-conjugated anti-mouse IgG (p0447; Dako, Glostrup, Denmark). Relative intensities of bands were quantified by densitometric analyses using Image Studio Digits Version 3.1 (LI-COR).

### Mice

Female Balb/cAnNRj (8 weeks old) mice were purchased from Janvier Labs (Le Genest-Saint-Isle, France), and housed in animal facilities at 19-25° C. with 12-hour light/dark cycles and free access to laboratory rodent diet and water. The mice had at least 1 week acclimation period before the experiments started.

### Atopic Dermatitis Mouse Model

Mice were randomly divided to receive: vehicle and drug-vehicle (negative control); MC903 (MedChemExpress, Monmouth Junction, NJ, USA) and drug-vehicle (disease control); MC903 and topical or oral Compound I (Compound I treatment groups); or MC903 and topical dexamethasone (positive control; purchased from Sigma-Aldrich). Four to eight mice were allocated to each group, totalling 76 mice for all experiments.

To induce AD-like skin inflammation, 1 or 2.5 nmol of MC903 in 25 μL absolute ethanol was topically applied once daily to the ventral and dorsal surface of the right ear for six consecutive days. After MC903 challenge, the mice were treated with topical Compound I (1 mg in 25 μL ethanol), oral Compound I (100 mg/kg), topical dexamethasone (20 nmol in 25 μl ethanol), or drug-vehicle (topical ethanol or oral 5% kleptose [HPB, Roquette Pharma, Lestrem, France]). The topical treatments were applied 2 hours post-MC903 challenge, whereas the oral treatments were administered by oral gavage immediately post-MC903 challenge. At the end of the experiment on day 9, clinical photos of the ears were taken and the mice were euthanised by cervical dislocation. Two punch biopsies (4 mm) from the ear were collected for RNA isolation and histological assessment. Ear thickness, the primary clinical endpoint for inflammation, was measured daily with a digital calliper (Mitutoyo Corporation, Kawasaki, Japan), and body weight was measured throughout the study to monitor the welfare of mice. During the experimental procedures, the mice were anesthetized with 2% isoflurane.

### Histology

Four-millimetres punch biopsies from the ear were fixed in 4% formaldehyde overnight at 4° C., paraffin-embedded, and serially sliced into 4 μm sections subjected to staining procedures. For haematoxylin & eosin (HE) staining, a standard protocol was followed. To detect mast cells, sections were stained with 0.1% toluidine blue solution (Sigma-Aldrich) at pH 2.3 and room temperature for 3 minutes. For immunohistochemical staining, heat-induced antigen retrieval was performed (25 minutes at 97° C.) in Tris-EGTA buffer (pH 9), and stained with anti-CD4 antibody (1:1000; EPR19514, Abcam, Cambridge, UK) or anti-Ly6g antibody (1:2000; EPR22909-135, Abcam) for 1 hour at room temperature. The remaining steps for detection were performed with Ultravision Quanto Detection System (ThermoFisher) following the manufacturer's instructions. The sections were counterstained with hematoxylin.

All slides were scanned with 20× objective using the whole slide scanner NanoZoomer 2.0-HT (Hamamatsu Photonics K.K, Hamamatsu City, Japan). Quantitative image analysis was performed in QuPath 0.4.2 using the Cell Detection command with default settings followed by a manual review and corrections.¹⁸ The number of counted cells was normalised to the length of the section.

### RNA Sequencing

Paired-end RNA sequencing of total RNA was conducted by Eurofins Genomics Europe Sequencing GmbH (Konstanz, Germany) in accordance with their protocols. To prepare the RNA library, NEBNext Ultra II Directional RNA Library Prep Kit for Illumina was used with 100 ng of total RNA. The mRNA quality was assessed by Fragment Analyzer. Illumina NovaSeq 6000 platform in 2×150 Sequence mode was performed to acquire at least 20 million read pairs. Read alignment to the mouse reference genome (GRCm39 primary assembly) and counting of uniquely aligned unambiguous reads were performed with the R package Subread (version 2.10.3).¹⁹ Regularized logarithm transformation (rlog) performed with the R package DESeq2 (1.36.0) was used for library size correction and variance stabilization.²⁰ The rlog normalized counts were used for principal component analysis (PCA) and heat map analysis (pheatmap 1.0.12). DESeq2 with independent filtering and Wald test enabled were used for the differential gene expression analyses.²⁰ To adjust for multiple comparisons, the p-values were adjusted by setting the false discovery rate (FDR) at 0.05 with the Benjamini and Hochberg procedure. Kyoto Encyclopaedia of Genes and Genomes (KEGG) and Gene Ontology (GO) enrichment analyses were generated with ShinyGO 0.76 and validated with DAVID.^{21,22}

### Statistical Analysis

The statistical significance threshold was set at P<0.05. Differences between treatment groups in clinical endpoints, mRNA (qPCR) and protein levels (western blot) were analysed by unpaired t-tests for mice and paired t-tests for keratinocytes. If data were not normally distributed, Mann-Whitney and Wilcoxon signed-rank tests were performed. All statistical analyses were generated with GraphPad Prism 9.0 and R 4.2.0 software.

### Results

### Compound I Robustly Inhibited AD-Associated Cytokine Expression in NHEKs

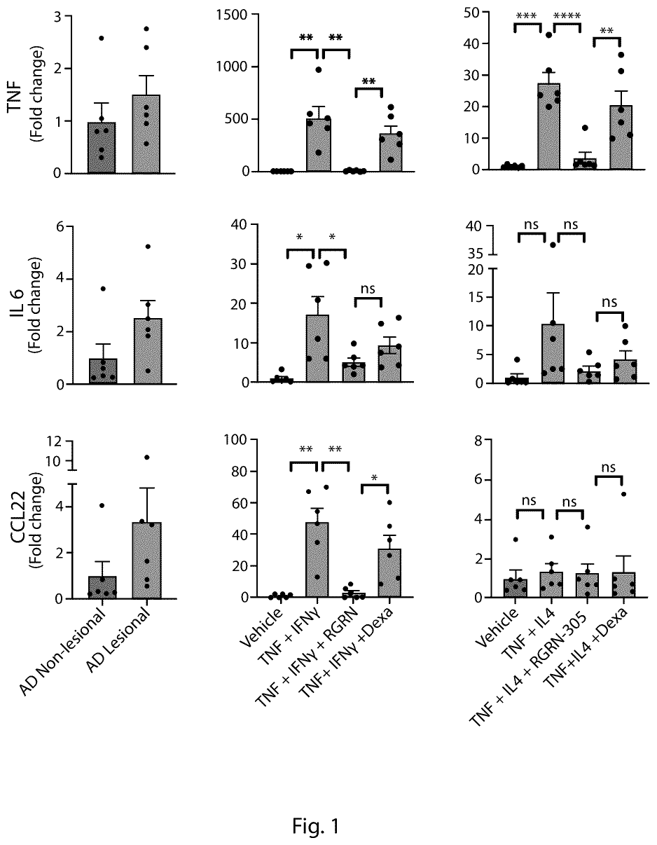

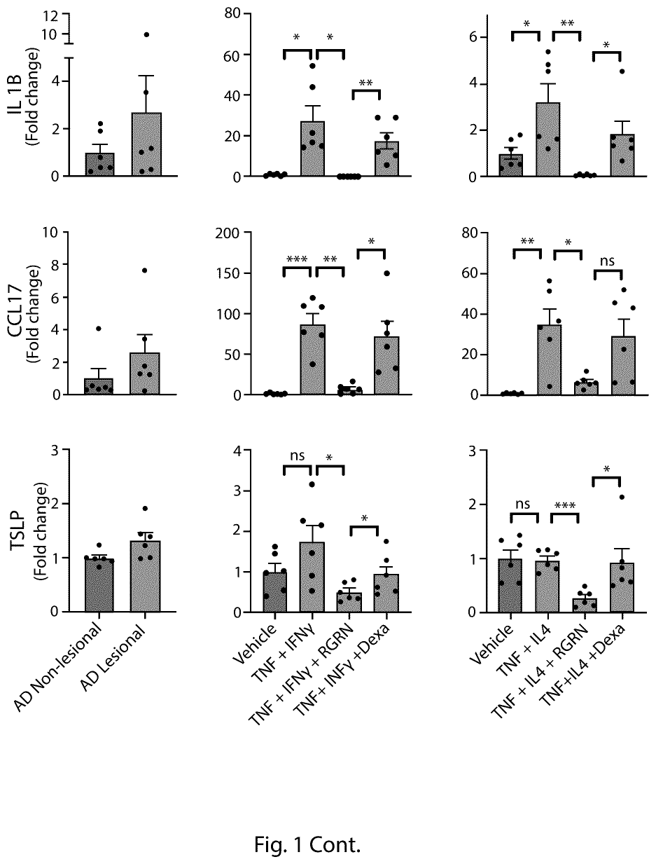

To explore the anti-inflammatory effects of HSP90 inhibition in atopic dermatitis, NHEKs from healthy donors were stimulated with TNF/IFNγ or TNF/IL-4, as experimental models, to mimic an AD-related gene expression. qPCR analysis demonstrated that inflammatory cytokines (TNF, IL1B, IL6) and chemokines (CCL17, CCL22) were statistically significantly upregulated in these models, consistent with an observed increased expression in human AD lesional skin compared with non-lesional skin (FIG. 1). Interestingly, Compound I robustly inhibited the expression of these AD-associated cytokines and chemokines compared with stimulated/vehicle- and stimulated/dexamethasone-treated NHEKs. While not significantly upregulated in our models, Compound I also suppressed the expression of TSLP, supporting a convincing anti-inflammatory effect mediated by HSP90 inhibition in keratinocytes.

### Compound I Suppresses the Activity of STAT3 and STAT6 Signalling Pathways in NHEKs

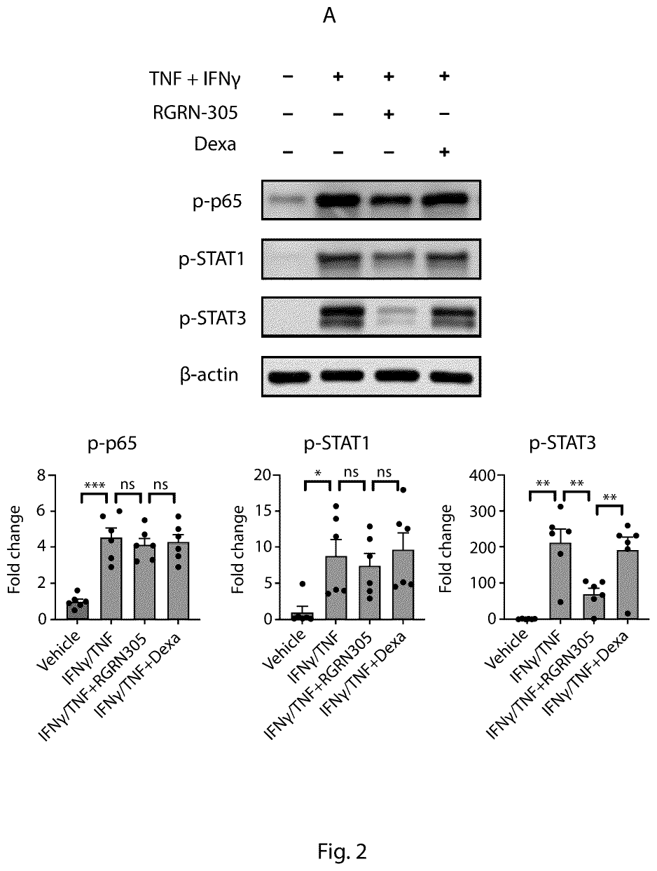

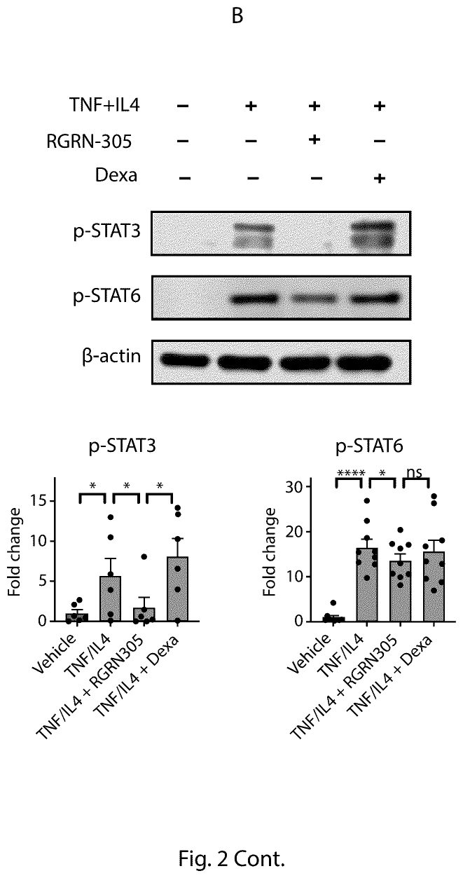

To further elucidate the potential AD-related pathways targeted by HSP90 inhibition in keratinocytes, the phosphorylation status of key signalling proteins, including STAT1, STAT3, STAT6 and p65 (subunit of NF-κB), was determined by Western blot (FIG. 2). Importantly, Compound I significantly suppressed the phosphorylation of STAT3 under TNF/IFNγ or TNF/IL-4 stimulation, and to a lesser degree STAT6 (~20% reduction) under TNF/IL-4 stimulation. However, Compound I had no significant effects on the phosphorylation of STAT1 and p65.

### Topical Compound I Attenuates MC903-Induced AD-Like Inflammation in Mice

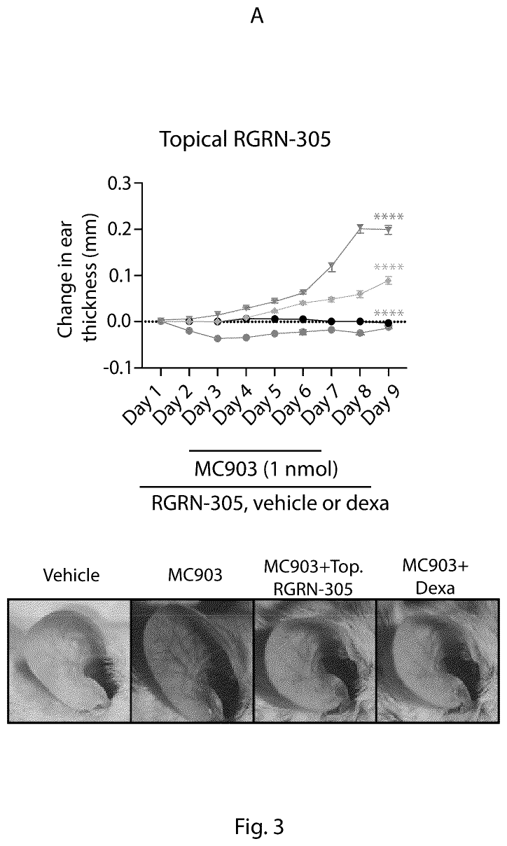

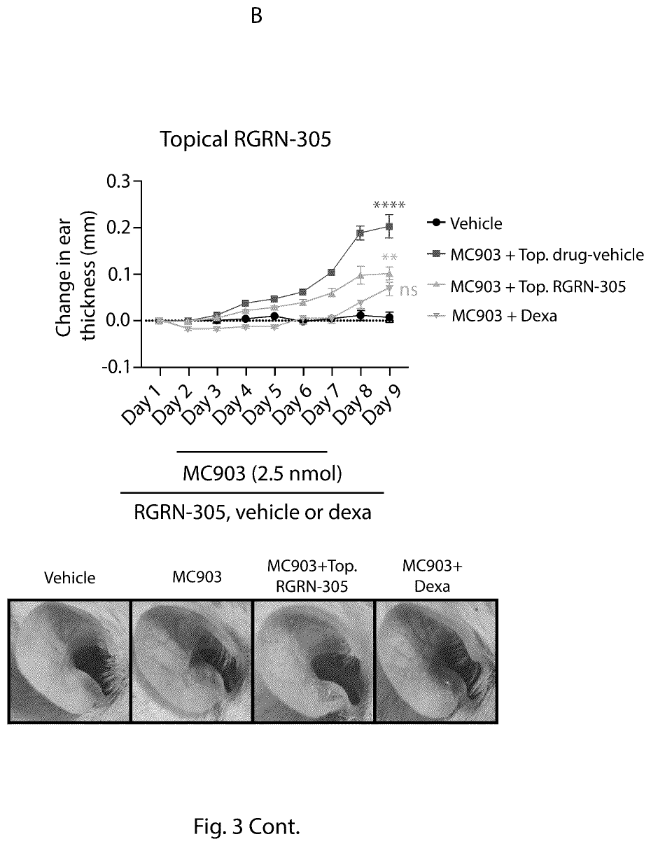

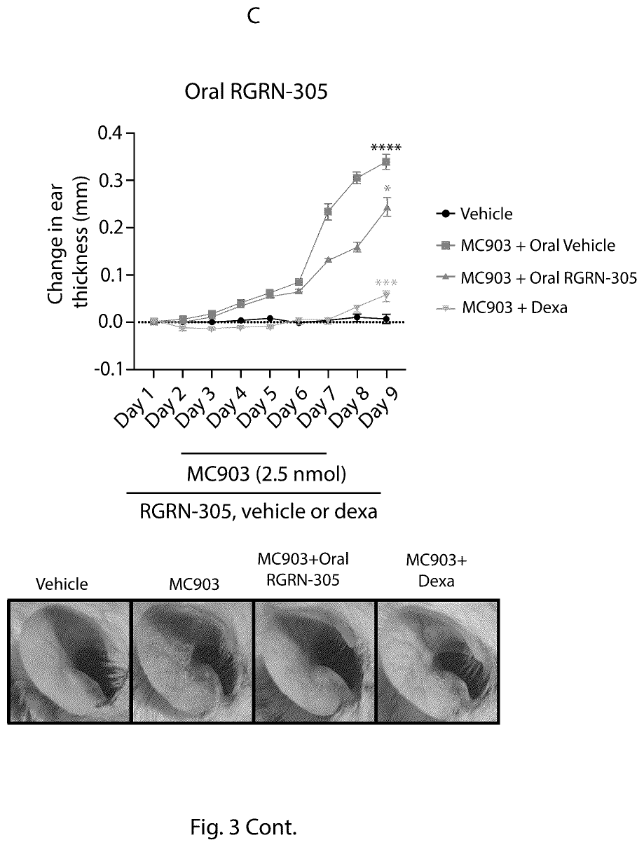

Next, the effects of HSP90 inhibition on AD-like skin inflammation were investigated in a mouse model for atopic dermatitis. The right ears of BALB/c mice were challenged daily with 1 nmol MC903 or vehicle for 6 days and received topical treatment with drug-vehicle (ethanol), Compound I or dexamethasone throughout the experiment for 9 days. Treatment with Compound I resulted in visibly reduced erythema and a highly significant reduction (55%) in ear thickness (a marker of inflammation) compared with drug-vehicle at the end of the experiment on day 9 (FIG. 3A). Dexamethasone treatment was highly potent resulting in ear thickness below baseline levels and more effective than Compound I. However, dexamethasone-treated mice lost significantly more weight than Compound I-treated mice, indicating a more favourable safety profile for Compound I.

Next, the experiment was repeated with 2.5 nmol (increased from 1 nmol) of MC903 challenge to observe whether Compound I could suppress a stronger stimulus of inflammation (FIG. 3B). Interestingly, topical Compound I still significantly suppressed the MC903-induced ear thickening by 50% on day 9, which was comparable to the dexamethasone treatment (P=0.13).

### Oral Compound I Ameliorates MC903-Induced AD-Like Inflammation in Mice

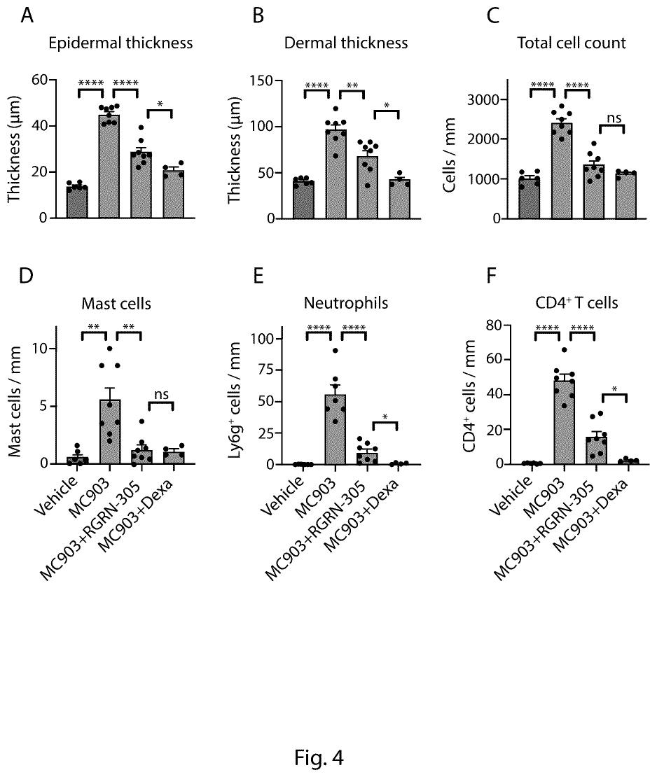

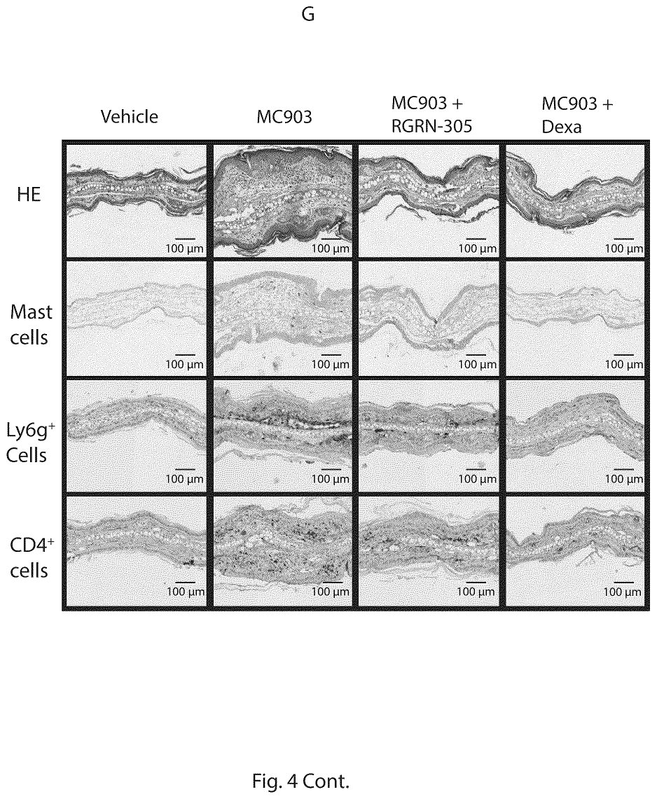

To evaluate the feasibility of orally administered Compound I, mice were challenged with 2.5 nmol MC903 for six days and administered 100 mg/kg Compound I by oral gavage once daily throughout the experiment. Oral Compound I treatment led to visibly reduced erythema and significantly reduced ear thickening (28% reduction) compared with drug-vehicle, but Compound I was less effective than dexamethasone (FIG. 4C). Furthermore, an independent testing facility (Comparative Biosciences, Sunnyvale, CA, USA) demonstrated a dose-dependent improvement by oral Compound I (20, 50 and 100 mg/kg) in alleviating AD-like inflammation in BALB/c mice using an alternative MC903 regimen.

### Topical Compound I Decreases MC903-Induced Immune Cell Infiltration in Mice

To gain insight into the mechanisms of HSP90, punch biopsies derived from the experiment with topical Compound I and 1 nmol MC903 challenge were investigated. Histological analyses confirmed that MC903 increased the epidermal and dermal thickness, which was significantly decreased by Compound I or dexamethasone (FIG. 4A-B). Moreover, the total number of cells within histological sections was significantly reduced by Compound I to levels broadly similar with dexamethasone (FIG. 4C). In line with this, Compound I significantly suppressed immune cell infiltration into the skin including mast cells, Ly6g+ cells (neutrophils), and CD4+ (T-cells; FIG. 4D-G).

### Topical Compound I Inhibits MC903-Induced Transcriptome Alterations and AD-Associated Cytokine Expression in Mice

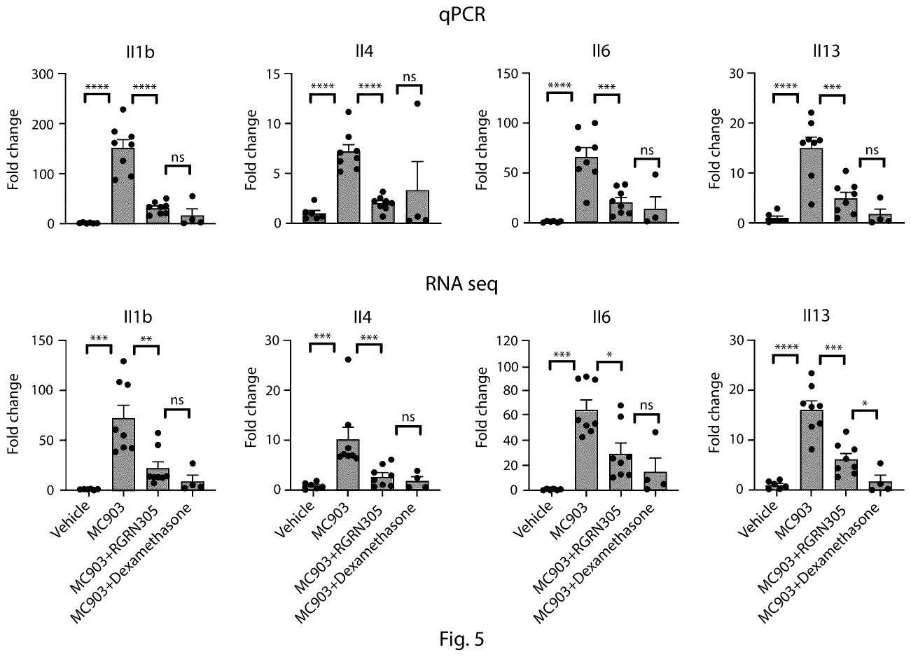

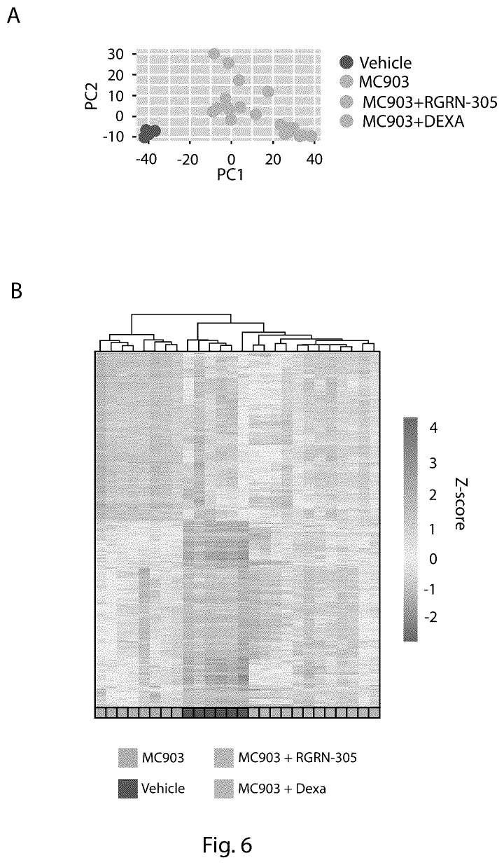

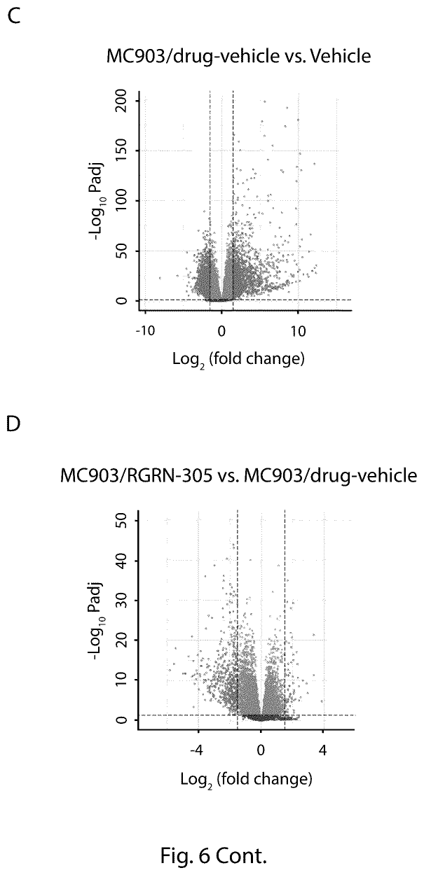

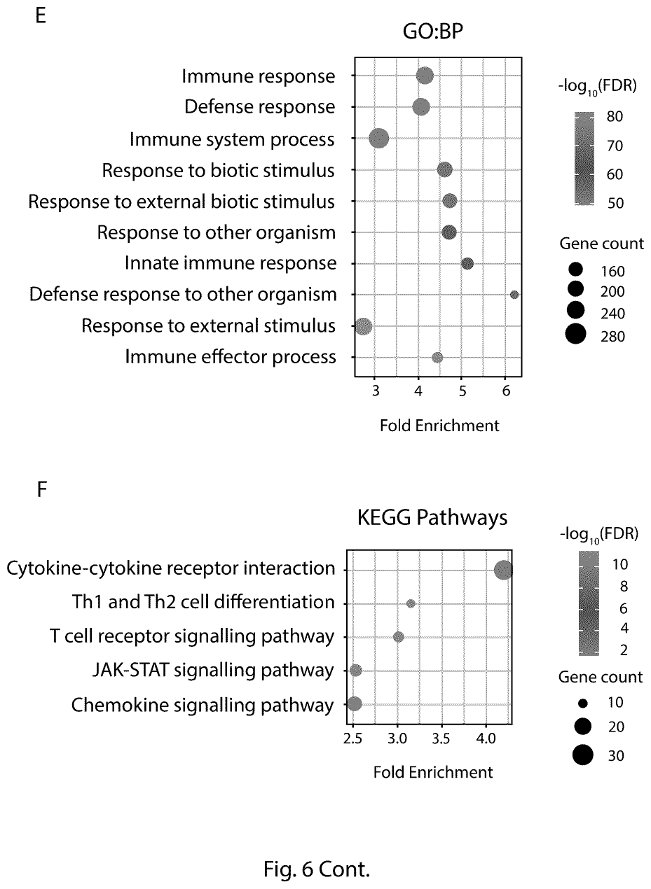

To examine alterations in gene expression, RNA sequencing and qPCR analyses of ear tissue were performed and showed that topical Compound I-treated mice exhibited significantly reduced cytokine expression of Il1b, Il4, Il6 and Il13 compared with drug-vehicle-treated mice (FIG. 5). Using RNA sequencing data, a principal component analysis (PCA) and a hierarchically clustered heatmap revealed that the mice clustered into their respective treatment groups (FIGS. 6A-B). The MC903/drug-vehicle-treated mice exhibited a distinctive expression pattern separated from Compound I-treated mice, indicating that Compound I treatment mitigates MC903-induced transcriptome alternations. In addition, volcano plots illustrate that MC903 challenge skewed the trend of differentially expressed genes (DEGs) to the right, indicating upregulation of genes (FIG. 6C); whereas, Compound I treatment compared with drug-vehicle, skewed the trend of DEGs to the left, indicating down-regulation of genes induced by MC903 (FIG. 6D). To explore the biological functions of the DEGs, enrichment analyses were performed for down-regulated DEGs by Compound I compared with drug-vehicle. The analyses revealed that the top ten significant GO biological process (BP) terms were mostly related to inflammation, revealing that Compound I suppressed genes involved in inflammation (FIG. 6E). Furthermore, KEGG pathway enrichment analysis showed that Compound I down-regulated genes implicated in inflammatory pathways including JAK-STAT signalling (FIG. 6F).

### DISCUSSION

The results of this study show that HSP90 inhibition by Compound I robustly suppressed inflammation in experimental models of AD by significantly reducing clinical symptoms of dermatitis (erythema and oedema), immune cell infiltrations, expression of key cytokines (e.g., IL-4 and IL-13) and signalling pathways (STAT3 and STAT6) related to AD. Thus, the encouraging results suggest HSP90 may be considered a novel therapeutic target for AD, providing preclinical validation for further clinical investigations in patients.

While HSP90 inhibitors have been researched for oncological disorders, the therapeutic potential of HSP90 inhibitors in inflammation has been sparsely examined. To the best of our knowledge, two proof-of-concept clinical studies and a small number of preclinical studies have been conducted.^{11,23,24} The findings are in line with other preclinical studies beyond atopic dermatitis wherein HSP90 inhibition exerted anti-inflammatory effects by targeting several inflammatory cytokines (e.g., TNF, IL-1B, and IL-6) and signalling pathways (e.g., ERK1/2, p38, and JNKs).^{8,25-27}

Atopic dermatitis is routinely treated with emollients together with topical anti-inflammatory treatments such as corticosteroids or calcineurin inhibitors as first-line therapies, whereas systemic treatments are reserved for more severe or recalcitrant cases.²⁸ We demonstrated that both topical and oral Compound I significantly reduced AD-like skin inflammation in mice. While both routes of administration may be feasible in treating AD, topically delivered Compound I may be the better option as the reduction of ear thickness was 50-54% compared with 28% for oral Compound I. Moreover, topical Compound I demonstrated similar or slightly inferior efficacy compared with topical dexamethasone, a very potent corticosteroid.²⁹ Yet, Compound I treatment resulted in significantly less weight loss compared with dexamethasone, indicating a more favourable safety profile. It may be worth noting that orally delivered Compound I may also require additional time—than this short duration study allowed (9 days)—to build sufficient levels in the skin. In agreement, oral treatment with Compound I has been well-tolerated in proof-of-concept studies with psoriasis and hidradenitis suppurative patients.¹¹ Furthermore, both topical and oral dose and frequency of Compound I may need to be optimized to achieve efficacy comparable to Dexamethasone, whilst maintaining its superior safety profile.

In the acute phase of AD, a Th2 response is initiated and shifts towards a dominancy of Th1 response as the disease progresses into its chronic stage.^{30,31} Activated keratinocytes, as key effector cells, play a crucial role in promoting immune dysregulation by secreting a variety of cytokines and chemokines (eg., IL-1β, IL-6, CCL17, TSLP), contributing to the pathogenesis in AD.³² In primary human keratinocytes and mice, Compound I treatment strongly suppressed Th1- and Th2-associated cytokines and chemokines implicated in AD (FIGS. 1 and 5). In human keratinocytes, Compound I inhibited the phosphorylation (i.e., activation) of STAT3 and, to a lesser extent, STAT6 (FIG. 2); two key signalling proteins downstream of the IL-4 receptor.³³ In accordance, enrichment analysis from the mouse model showed JAK-STAT signalling was highly downregulated by Compound I. Interestingly, another recent study (n=7) discovered that HSP90 inhibition disrupted JAK-STAT signalling by potently decreasing JAK2 expression and the phosphorylation of STAT3 and STAT5 in patients with myeloproliferative neoplasms, further supporting our findings.³⁴ The recent success of therapeutics targeting JAK-STAT pathways highlights the importance of these pathways in AD, and it may be one of the mechanisms by which HSP90 inhibition ameliorates AD.³⁵ However, considering the wide range of client proteins targeted by HSP90 inhibition, the anti-inflammatory effects are most likely attributable to other mechanisms as well.

Limitations of the study include the inherent nature of basic research and experimental models that may encounter challenges when translating the findings into clinical practice. Nonetheless, the accumulating evidence of HSP90 inhibition exerting broad anti-inflammatory effects together with the findings from this study increases the likelihood of clinical success in treating AD. Lastly, the permeability of mice skin varies to human skin, therefore further studies should evaluate optimal drug formulations enhancing the permeation and efficacy of topical Compound I.

In summary, HSP90 inhibition by Compound I potently suppressed inflammation using in vitro and in vivo experimental models mimicking AD, providing evidence that HSP90 inhibition is a novel mechanism of action in treating AD.

### Example 2—Pharmacokinetic Studies of Compound I

A 16-week treatment, randomized, double-blind, proof-of-concept study was designed to assess the safety and efficacy of Compound I compared to placebo in treating hidradenitis suppurativa. Eligible subjects completed an up-to 4-week screening phase and sequentially be randomized 2:1 to one of two cohorts, treatment cohort (n=10) or placebo-control cohort (n=5), in a 16-week treatment phase followed by a 4-week observational follow-up phase. In the 16-week treatment phase, subjects randomized to the treatment group receive 250 mg Compound I free base in the form of a tablet administered orally once daily. The 250 mg dose was selected based on the results of an earlier oncology trial conducted with doses in excess of 1000 mg/day as a safe minimal dose from which to conduct dose escalation if required. Subjects randomized to the placebo-control group receive placebo treatment once daily (microcrystalline cellulose, mannitol, crospovidone, Opadry II red, magnesium stearate, colloidal silicon dioxide).

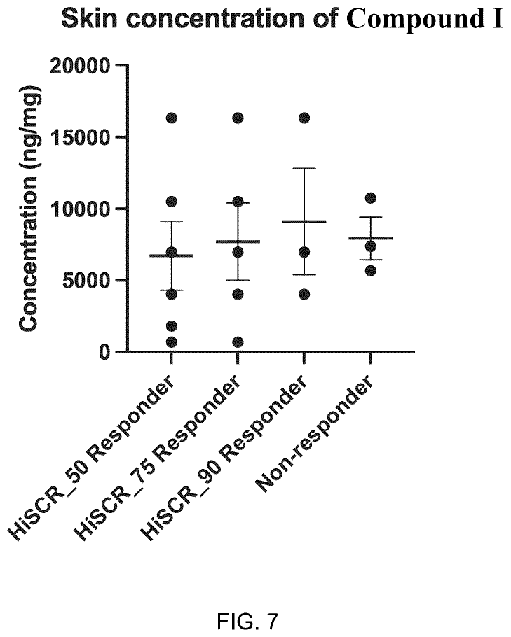

The accumulation of Compound I in the skin of human hidradenitis suppurativa of subjects in the treatment cohort was determined. Snap frozen skin punch biopsies obtained at the week 16 visit showed high concentrations of Compound I, indicating that Compound I accumulates in the skin. FIG. 7 is a graph showing the concentrations of Compound I found in the biopsied tissues in subjects grouped according to their clinical response to Compound I therapy. The mean skin concentration following 16 weeks of treatment with Compound I was 7130 ng/mg, which is equivalent to 16.1 mM.

The skin accumulation found in this study was compared to plasma levels of Compound I determined in a previous study in healthy human volunteers. Six healthy male subjects were given a single oral dose of 250 mg Compound I, and plasma samples were collected pre-dose and at 0.25, 0.5, 0.75, 1, 1.5, 2, 2.5, 3, 4, 6, 8, 10, 12, 18, 24, 30, 36, 48, 72 and 96 hours after dosing. The results of this study are shown in the table below.

| Subject | AUCₗₐₛₜ | AUC_{∞} |
|---|---|---|
| ID | (ng*hr/mL) | (ng*hr/mL) |
| 1 | 51.64 | 51.64 |
| 2 | 70.13 | 85.79 |
| 3 | 36.82 | 36.82 |
| 4 | 25.64 | 25.64 |
| 5 | 45.08 | 45.08 |
| 6 | 54.08 | 54.08 |

The Cₘₐₓ for Compound I determined for the six subjects ranged from 19.42 ng/ml to 24.92 ng/mL, with a median of 20.3 ng/ml or 46 nM.

The results show that the accumulation of Compound I is approximately 100,000-fold higher in the skin than in the plasma following daily oral dosing at 250 mg. In addition, the observed skin concentrations exceed the IC⁵⁰ of Compound I for inhibition of HSP-90 (0.1 μM) by a factor of about 10⁴. One of skill in the art would understand that a daily oral dose of Compound I which is significantly less than 250 mg, for example, as low as 1 mg per day, will result in sufficient skin accumulation of Compound I to inhibit HSP-90 in the skin and thereby treat atopic dermatitis.

### REFERENCES

1. Langan S M, Irvine A D, Weidinger S. Atopic dermatitis. Lancet (London, England) 2020; 396(10247): 345-60.
2. Barbarot S, Auziere S, Gadkari A, et al. Epidemiology of atopic dermatitis in adults: Results from an international survey. Allergy 2018; 73(6): 1284-93.
3. Silverberg J I, Barbarot S, Gadkari A, et al. Atopic dermatitis in the pediatric population: A cross-sectional, international epidemiologic study. Annals of allergy, asthma & immunology: official publication of the American College of Allergy, Asthma, & Immunology 2021; 126(4): 417-28.e2.
4. Silverberg J I. Comorbidities and the impact of atopic dermatitis. Annals of allergy, asthma & immunology: official publication of the American College of Allergy, Asthma, & Immunology 2019; 123(2): 144-51.
5. Ben Abdallah H, Vestergaard C. Atopic dermatitis, hypertension and cardiovascular disease. British Journal of Dermatology 2022; 186(2): 203-4.
6. Kim J, Kim B E, Leung DYM. Pathophysiology of atopic dermatitis: Clinical implications. Allergy Asthma Proc 2019; 40(2): 84-92.
7. Bieber T. Atopic dermatitis: an expanding therapeutic pipeline for a complex disease. Nat Rev Drug Discov 2022; 21(1): 21-40.
8 Ben Abdallah H, Seeler S, Bregnhøj A, et al. Heat shock protein 90 inhibitor RGRN-305 potently attenuates skin inflammation. Frontiers in immunology 2023; 14:1128897.
9. Stenderup K, Rosada C, Gavillet B, Vuagniaux G, Dam T N. Debio 0932, a new oral Hsp90 inhibitor, alleviates psoriasis in a xenograft transplantation model. Acta Derm Venereol 2014; 94(6): 672-6.
10. Hansen R S, Thuesen K K H, Bregnhøj A, et al. The HSP90 inhibitor RGRN-305 exhibits strong immunomodulatory effects in human keratinocytes. Experimental dermatology 2021.
11. Bregnhøj A, Thuesen KKH, Emmanuel T, et al. HSP90 inhibitor RGRN-305 for oral treatment of plaque-type psoriasis: efficacy, safety and biomarker results in an open-label proof-of-concept study. The British journal of dermatology 2022; 186(5): 861-74.
12. Sitko K, Bednarek M, Mantej J, Trzeciak M, Tukaj S. Circulating heat shock protein 90 (Hsp90) and autoantibodies to Hsp90 are increased in patients with atopic dermatitis. Cell Stress Chaperones 2021; 26(6): 1001-7.
13. Johansen C. Generation and Culturing of Primary Human Keratinocytes from Adult Skin. J Vis Exp 2017; (130).
14. Kim H J, Baek J, Lee J R, Roh J Y, Jung Y. Optimization of Cytokine Milieu to Reproduce Atopic Dermatitis-related Gene Expression in HaCaT Keratinocyte Cell Line. Immune Netw 2018; 18(2): e9.
15. Han J, Cai X, Qin S, et al. TMEM232 Promotes the Inflammatory Response in Atopic Dermatitis via NF-κB and STAT3 Signaling Pathways. The British journal of dermatology 2023.
16. Piazza S, Martinelli G, Magnavacca A, et al. Unveiling the Ability of Witch Hazel (Hamamelis virginiana L.) Bark Extract to Impair Keratinocyte Inflammatory Cascade Typical of Atopic Eczema. International journal of molecular sciences 2022; 23(16).
17. Biosystems A. Applied Biosystems StepOne™ and StepOnePlus™ Real-Time PCR Systems. Applied Biosystems; 2010.
18. Bankhead P, Loughrey M B, Fernández J A, et al. QuPath: Open source software for digital pathology image analysis. Sci Rep 2017; 7(1): 16878.
19. Liao Y, Smyth G K, Shi W. The R package Rsubread is easier, faster, cheaper and better for alignment and quantification of RNA sequencing reads. Nucleic Acids Res 2019; 47(8): e47.
20. Love M I, Huber W, Anders S. Moderated estimation of fold change and dispersion for RNA-seq data with DESeq2. Genome Biol 2014; 15(12): 550.
21. Ge S X, Jung D, Yao R. ShinyGO: a graphical gene-set enrichment tool for animals and plants. Bioinformatics 2020; 36(8): 2628-9.
22. Sherman B T, Hao M, Qiu J, et al. DAVID: a web server for functional enrichment analysis and functional annotation of gene lists (2021 update). Nucleic Acids Res 2022; 50(W1): W216-w21.
23. Tukaj S, Sitko K. Heat Shock Protein 90 (Hsp90) and Hsp70 as Potential Therapeutic Targets in Autoimmune Skin Diseases. Biomolecules 2022; 12(8).
24. Tukaj S, Węgrzyn G. Anti-Hsp90 therapy in autoimmune and inflammatory diseases: a review of preclinical studies. Cell Stress Chaperones 2016; 21(2): 213-8.
25. Rice J W, Veal J M, Fadden R P, et al. Small molecule inhibitors of Hsp90 potently affect inflammatory disease pathways and exhibit activity in models of rheumatoid arthritis. Arthritis and rheumatism 2008; 58(12): 3765-75.
26. Yun T J, Harning E K, Giza K, et al. EC144, a synthetic inhibitor of heat shock protein 90, blocks innate and adaptive immune responses in models of inflammation and autoimmunity. J Immunol 2011; 186(1): 563-75.
27. Nizami S, Arunasalam K, Green J, et al. Inhibition of the NLRP3 inflammasome by HSP90 inhibitors. Immunology 2021; 162(1): 84-91.
28. Flohr C. How we treat atopic dermatitis now and how that will change over the next 5 years. The British journal of dermatology 2022.
29. Johnson D B, Lopez M J, Kelley B. Dexamethasone. StatPearls. Treasure Island (F L): StatPearls Publishing Copyright © 2023, StatPearls Publishing LLC.; 2023.
30. Bieber T. Atopic dermatitis. The New England journal of medicine 2008; 358(14): 1483-94.
31. Guttman-Yassky E, Krueger J G, Lebwohl M G. Systemic immune mechanisms in atopic dermatitis and psoriasis with implications for treatment. Experimental dermatology 2018; 27(4): 409-17.
32. Das P, Mounika P, Yellurkar M L, et al. Keratinocytes: An Enigmatic Factor in Atopic Dermatitis. Cells 2022; 11(10).
33. Dubin C, Del Duca E, Guttman-Yassky E. The IL-4, IL-13 and IL-31 pathways in atopic dermatitis. Expert review of clinical immunology 2021; 17(8): 835-52.
34. Hobbs G S, Hanasoge Somasundara A V, Kleppe M, et al. Hsp90 inhibition disrupts JAK-STAT signaling and leads to reductions in splenomegaly in patients with myeloproliferative neoplasms. Haematologica 2018; 103(1): e5-e9.
35. Huang I H, Chung W H, Wu P C, Chen C B. JAK-STAT signaling pathway in the pathogenesis of atopic dermatitis: An updated review. Frontiers in immunology 2022; 13:1068260.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts, and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method for treating atopic dermatitis in a subject in need thereof, comprising administering to the subject a therapeutically effective amount of Compound I, 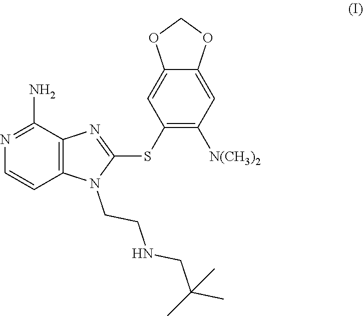 [SMILES: CN(C)c1cc2c(cc1Sc1nc3c(N)nccc3n1CCNCC(C)(C)C)OCO2]
or a pharmaceutically acceptable salt thereof, wherein the compound or pharmaceutically acceptable salt is orally administered at a free base equivalent dose of 0.5 mg to 400 mg.

2. (canceled)

3. The method of claim 1, wherein the compound or a pharmaceutically salt thereof is administered to the subject from 3 to 7 days per week.

4. (canceled)

5. The method of claim 1, wherein Compound I is administered orally to the subject as the free base at a dose of 0.5 mg to 400 mg per day.

6. The method of claim 1, wherein Compound I is administered orally to the subject as the free base at a dose of 25 mg to 275 mg per day, 30 mg to 260 mg per day, 50 to 255 mg per day or 250 mg per day.

7. The method of claim 1, wherein a pharmaceutically acceptable salt of Compound I is administered orally to the subject at a free base equivalent dose of 200 mg to 300 mg per day.

8. The method of claim 7, wherein the free base equivalent dose is 225 mg to 275 mg per day, 240 mg to 260 mg per day, 245 to 255 mg/day or 250 mg per day.

9. The method of claim 1, wherein Compound I or pharmaceutically acceptable salt thereof, is orally administered in the form of a tablet.

10. The method of claim 9, wherein the tablet comprises from 10 mg to 350 mg, from 20 mg to 300 mg, from 40 mg to 275 mg, 50 mg to 260 mg or 250 mg free base equivalent amount of Compound I or a pharmaceutically acceptable salt thereof.

11.-13. (canceled)

14. The method of claim 6, wherein Compound I is administered to the subject as the free base at a dose of 25 mg to 400 mg per day, 30 mg to 300 mg per day, 50 to 255 mg/day or 250 mg per day.
